(19) United States
(12) Patent Application Publication
(10) Pub. No.: US 2024/0368067 A1
(43) Pub. Date: Nov. 7, 2024

(54) IMPROVED METHOD FOR PRODUCING HIGH-PURITY BUTYL ACRYLATE

(71) Applicant: Arkema France, Colombes (FR)

(72) Inventors: Serge TRETJAK, Saint Avold (FR); Andre LEVRAY, Saint Avold (FR)

(21) Appl. No.: 18/291,680

(22) PCT Filed: Jul. 25, 2022
(86) PCT No.: PCT/FR2022/051488

§ 371 Date: Jan. 24, 2024

(30) Foreign Application Priority Data
Aug. 25, 2021 (FR) FR21.08885

(51) Int. Cl. C07C 67/08; C07C 67/54

(52) U.S. Cl. CPC C07C 67/08; C07C 67/54

(57) ABSTRACT

A process for the production of butyl acrylate by direct esterification of acrylic acid with butanol, the reaction being catalyzed by sulfuric acid. More specifically, the present invention relates to an improved process for producing butyl acrylate comprising a step of upgrading the heavy by-products generated during this production, leading to a high productivity of a product meeting the standards as regards purity and acidity, under optimized energy conditions.

### TECHNICAL FIELD

The present invention concerns the production of butyl acrylate by direct esterification of acrylic acid with butanol, this reaction being catalyzed by sulfuric acid. More specifically, it relates to an improved process for producing butyl acrylate, comprising a step of upgrading the heavy by-products generated during this production, leading to a high productivity of a product meeting the standards as regards purity and acidity, under optimized energy conditions.

### TECHNICAL BACKGROUND AND PROBLEM

The esterification of acrylic acid is a balanced reaction with generation of water that must be removed during the reaction to shift the equilibrium in the direction of production of the acrylic ester.

The problems that arise in the production of butyl acrylate by direct esterification of acrylic acid, generally in the presence of sulfuric acid as catalyst, are most often related to the complexity of the purification steps necessary after the reaction step to obtain a product of high purity, to the detriment of the productivity of the process.

The industrial process, as described in patent EP 609127 of the applicant, consists in esterifying acrylic acid with excess butanol, in the presence of sulfuric acid. The reaction mixture at the end of reaction comprises butyl acrylate, residual acrylic acid, butyl hydrogen sulfate, traces of sulfuric acid and various impurities resulting from side reactions. This reaction mixture is then subjected to a step of neutralization and washing with water, the object of which is to remove the so-called acidic impurities: residual sulfuric acid, butanol hydrogen sulfate and acrylic acid. This mixture, free of acidic impurities, is subjected to various purification steps, which lead to the recovery of the purified butyl acrylate. One of the stages, referred to as topping, consists in particular in distilling the butanol and the light by-products. Butanol can thus be recycled to the esterification reaction.

The final stage of purification of butyl acrylate consists in sending the mixture containing the ester from which the light products have been removed to a last distillation column from which it exits at the top, purified of the heavy by-products which are found at the bottom of the distillation column and are then concentrated in an evaporator.

Among the by-products generated according to the side reactions, mention may be made of light products such as butyl acetate, butyl propionate, dibutyl ether, isobutyl acrylate or heavy products such as dibutyl maleate.

The “heavy” compounds resulting from Michael addition reactions form spontaneously in butyl acrylate production units. These parasitic reactions are promoted by high temperatures encountered in particular in the bottoms of distillation columns of these units. Thus acrylic acid, yet unreacted butanol or water of reaction are added to the double bond of the butyl acrylate to form mainly:
butyl acryloxypropionate (AA/ABU) by addition of acrylic acid (AA) to butyl acrylate (ABU);
butyl hydroxypropionate (HPB) by addition of water to butyl acrylate;
butyl butoxypropionate (BPB) by addition of butanol to butyl acrylate.

Polyaddition or the formation of mixed compounds is also possible.

One of the characteristics of heavy by-products is that their boiling point is above the boiling points of acrylic acid, butanol and butyl acrylate. Since their volatility is low, they accumulate at the bottom of the last distillation column, at the bottom of the evaporator used to concentrate this residue.

The evaporator residue, in addition to the Michael derivatives and a few percent of free monomers, also contains a high concentration of polymerization inhibitors, accumulated during the purification steps, such as phenothiazine in its free form or as an adduct of AA or ABU, and also heavy compounds of polymeric nature which are more or less soluble in the medium. In general, this residue is removed by incineration, which leads to a significant loss of yield.

Various solutions have been proposed for the upgrading of these heavy by-products. Document CN1063678 proposes methods of treating the oxy-esters formed during the synthesis of butyl acrylate using protic acid catalysts such as sulfuric acid or para-toluenesulfonic acid. Compounds such as phthalates can also be added, as described in document U.S. Pat. No. 4,293,347.

The disadvantage of these cracking methods is that the residual product is viscous and contains solids. U.S. Pat. No. 6,617,470 proposes the use, as catalysts, of arylsulfonic acids such as dodecylsulfonic acid, which suppresses the formation of solids in the bottom residue.

Document US 2011/0230675 proposes adding water continuously when cracking is carried out by acid catalysis, in order to avoid the formation of solid deposits.

Document FR 2901272 proposes carrying out distillation before cracking, which limits the deposits of solid materials in the installations.

The applicant company has described in its patent FR 2727964 or in its application PCT/FR2021/050825 a thermal cracking of a mixture of acrylic acid (AA) by-products and acrylic esters with a view to their recycling in the acrylic ester production plant. It uses the property of alkoxy-propionic derivatives containing C—O—C bonds, such as ethyl 3-ethoxypropionate, of being more difficult to break than the C—C bonds of heavy by-products derived from acrylic acid.

The applicant company has described in its application FR2101402 a process diagram combining a side draw column, a thermal or thermal and catalytic cracker and a decanter and washing system for treating the products obtained from the cracker for the synthesis of acrylic ester such as 2-ethylhexyl acrylate. This combination is made necessary because, in these processes catalyzed by acid resins, the so-called acidic impurities first are not removed during a neutralization operation prior to the purification section and second are also generated during cracking. Without the combination of side draw column-decanter (see example 2), it is not possible to obtain a purified 2-ethylhexyl acrylate meeting the specifications. However, the transposition of the process diagram as envisaged in document FR2101402, for the synthesis of butyl acrylate, would lead to losses of upgradable materials resulting from cracking, such as acrylic acid, and especially butanol, which will be partially dissolved in water, when washing the cracker overhead product in the decanter.

Document JP 2015/140336 describes a process for producing butyl acrylate by direct esterification of acrylic acid with butanol in the presence of acid as catalyst, resulting in production of a crude reaction mixture containing butyl acrylate, acrylic acid, residual butanol and impurities.

Document FR 3032198 describes a process for the preparation of alkyl (meth)acrylate by transesterification followed by purification steps by distillation.

Finally, document CN102173990 proposes adding copper salts to the cracker feed in order to facilitate the subsequent treatment of the final cracking residue.

There is therefore still a need to improve the known processes for producing butyl acrylate, in order to obtain upgradable products as much as possible: acrylic acid, butanol and butyl acrylate, while avoiding the problems of solid formation. Moreover, it is desirable to limit the formation of by-products which could be difficult to separate in the purification steps leading to the production of purified butyl acrylate. These purification steps are carried out by distillation. Table 1 comparing the boiling temperatures at atmospheric pressure of the light by-products formed in the reaction makes it possible to identify the impurities whose formation will absolutely need to be limited during this cracking.

TABLE 1

| | Boiling temperature (° C.) |
|---|---|
| By-products formed | |
| Butyl acetate | 126 |
| Dibutyl ether | 141 |
| Butyl propionate | 146 |
| Isobutyl acrylate | 132 |
| Reagents and products | |
| Butyl acrylate | 148 |
| Acrylic acid | 141 |
| Butanol | 118 |

It has now been discovered that the implementation of a thermal cracking without catalyst makes it possible to transform Michael adducts with a high yield, without solid deposits in the installation, while limiting in particular the formation of dibutyl ether, in a process for producing high-purity butyl acrylate.

### SUMMARY OF THE INVENTION

The present invention describes a heat treatment making it possible to upgrade the Michael adducts in a process for producing butyl acrylate as described in patent EP 609127 for the reaction part.

It also completes the purification scheme described in this patent by associating a reactor for thermal cracking of the purge products from the bottom of the evaporator placed at the bottom of the butyl acrylate purification column and indicates the recycling of the overhead products obtained from cracking in the process.

It is based on two assumptions:
it is the interaction between phenothiazine and the acid catalyst that is largely responsible for the formation of coke during this cracking reaction, and
the amount of dibutyl ether increases when cracking is carried out in the presence of catalyst, which accentuates the butanol dehydration reaction.

The invention relates to a process for producing butyl acrylate by direct esterification of acrylic acid with excess butanol in the presence of sulfuric acid as catalyst, resulting in production of a crude reaction mixture containing butyl acrylate, residual acrylic acid and residual butanol, butyl hydrogen sulfate, traces of sulfuric acid and impurities resulting from side reactions, said process comprising steps of neutralization and washing with water leading to the production of a reaction mixture free of so-called acidic impurities, characterized in that said reaction mixture washed of acidic impurities is subjected at least to the following steps i), ii) and iii):
i) topping in a distillation column to obtain:
   at the top, a stream composed essentially of unreacted reagents;
   at the bottom, a stream comprising the desired ester and heavy by-products;
ii) the bottom stream from the topping column is subjected to a rectification column to separate:
   at the top, the purified desired ester;
   at the bottom, a stream containing heavy by-products, which is concentrated on a film evaporator or distilled in a tailing column in order to recycle the light compounds present to the rectification column, and to remove a final residue of heavy by-products;
iii) the bottom stream from the rectification column is subjected to a heat treatment carried out in the absence of catalyst in a cracker placed at the outlet of the evaporator, to separate:
   at the top, a stream of upgradable products recycled to the topping column feed;
   at the bottom, a residue sent to a treatment plant.

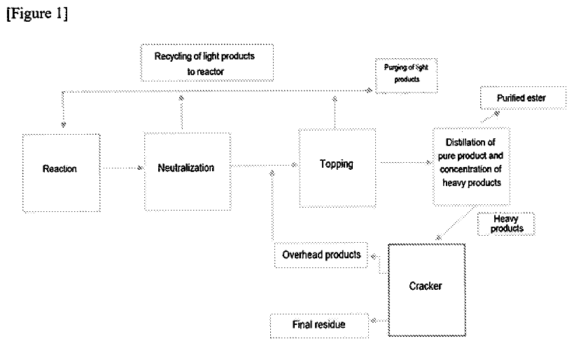

The present invention makes it possible to overcome the disadvantages of the state of the art. More particularly, it provides a process for obtaining a high-purity butyl acrylate having as specifications an ester purity greater than 99.5%, a dibutyl ether content of less than 500 ppm, and finally a water content of less than 400 ppm, incorporating a thermal process to crack the Michael adducts into reactants (acrylic acid and alcohol) and into finished product, thus increasing the productivity of the process by limiting the amount of residue to be removed. Other characteristics and advantages of the invention will become more apparent on reading of the detailed description which follows, with reference to the appended FIG. 1.

[FIG. 1]: overall diagram of the process for synthesis of butyl acrylate according to the invention, incorporating a thermal cracking step.

### DETAILED DESCRIPTION OF THE INVENTION

The invention relates to a process for producing butyl acrylate by direct esterification of acrylic acid with excess butanol, in the presence of sulfuric acid as catalyst, resulting in production of a crude reaction mixture containing butyl acrylate, residual acrylic acid and residual butanol, butyl hydrogen sulfate, traces of sulfuric acid and impurities resulting from side reactions.

According to various implementations, said process comprises the following features, where appropriate in combination.

After the esterification step, the process according to the invention comprises steps of neutralization and washing with water leading to the production of a reaction mixture freed of the so-called acidic impurities.

Acidic impurities generally include sulfuric acid, butyl hydrogen sulfate, acrylic acid dimer and residual acrylic acid.

According to one embodiment, the esterification step is followed by the addition to said crude reaction mixture of a base to neutralize the acrylic acid, butyl hydrogen sulfate, and traces of sulfuric acid present therein, the resulting salts passing into the aqueous phase of said mixture, the organic phase and the aqueous phase resulting from this neutralization being separated and the desired butyl acrylate being recovered from said organic phase.

The butyl acrylate recovery stage is also carried out in a conventional manner by washing with water, in an extraction column, the organic phase resulting from the phase separation which follows the first neutralization.

Typically, the reaction mixture washed of acidic impurities as described above is subjected at least to the following steps i), ii) and iii):
i) topping in a distillation column to obtain:
   at the top, a stream composed essentially of unreacted reagents;
   at the bottom, a stream comprising the desired ester and heavy by-products;
ii) the bottom stream from the topping column is subjected to a rectification column to separate:
   at the top, the purified desired ester;
   at the bottom, a stream containing heavy by-products, which is concentrated on a film evaporator or distilled in a tailing column in order to recycle the light compounds present to the rectification column, and to remove a final residue of heavy by-products;
iii) the bottom stream from the rectification column is subjected to a heat treatment carried out in the absence of catalyst in a cracker placed at the outlet of the evaporator, to separate:
   at the top, a stream of upgradable products recycled separately to the topping column feed;
   at the bottom, a residue sent to a treatment plant.

According to one embodiment, a thermal cracker is placed at the bottom of a purification column making it possible to obtain butyl acrylate at the top thereof and at the bottom the heavy products, which will first be concentrated on an evaporator. This concentrated stream is used to feed the thermal cracker. Optionally, without this harming the operation of the cracker, prior treatments of the feed stream, such as a distillation as described in patent FR2901272, can be implemented. The stream of upgradable products is recycled to the feed of the section, allowing the recycling of the alcohol to the reaction. The cracker bottom residue is sent to a treatment plant.

In this invention, the decomposition of the Michael adducts can be carried out in a continuous, semi-continuous or batch mode. The continuous mode is preferable because it corresponds to the preferred operation of this esterification process. It is possible to use a tubular reactor, a jacketed stirred reactor or a reactor having an external heating loop with forced circulation. The upgradable compounds generated by the cracking reaction are collected after condensation of the vapors at the top of the reactor or at the top of a distillation column surmounting this reactor.

The reaction temperature and pressure above the reactor are connected so that the reactants such as acrylic acid, butanol or the end product are removed by evaporation while butyl butoxypropionate (BPB), which is the main compound (>70% by weight) present in the cracker feed, is held in the reaction medium.

According to one embodiment, the decomposition reaction is carried out in a temperature range of 220° C. to 300° C. and more especially between 230° C. to 280° C.

According to one embodiment, the pressure maintained above the reactor is between 50 000 Pa to 300 000 Pa.

According to one embodiment, the mass composition of the cracker feedstock in the case of the production of butyl acrylate in the presence of phenothiazine as polymerization inhibitor is as follows:
Butanol <0.1%
Butyl acrylate (5-10%)
Butyl hydroxypropionate (HPB): 1-3%
Butyl butoxypropionate (BPB) 70-80%
Butyl acryloxypropionate (AA/ABU) 4-6%
Dibutyl maleate: 2-5%
Phenothiazine: 1-3%.

Advantageously, the heat treatment is carried out in the absence of catalyst.

The residence time in the cracker based on the feed flow rate (kg/h) relative to the volume of the liquid phase in the reactor is preferably chosen between 0.5 to 20 hours, and especially between 7 and 15 hours.

With reference to FIG. 1, which represents the preferred mode of the invention, the topping section comprises a distillation column having an equivalent of 10 and 30 theoretical trays, preferably 10 to 15 theoretical stages. The inserts used for the column may be valve trays or perforated weir trays, crossflow trays such as Dual Flow Trays, Ripple Trays, Turbo Grid Shells, or ordered packing, for instance structured packing such as Mellapack 250X from Sulzer.

The topping column is fed in the upper third of this column, preferably between the theoretical trays 3 to 10 counted from the top of the column. The top stream of the column essentially comprises the unreacted reagents. This upgradable stream is recycled to the reaction.

The column operates with a reflux ratio (flow rate of condensed liquid returned to the column/flow rate recycled to the reaction) of between 4/1 to 1/1, preferably 3/1. Advantageously, from 50 to 5000 ppm of polymerization inhibitor are introduced into the purification system according to the process of the invention.

As polymerization inhibitors which can be used, mention may be made, for example, of phenothiazine (PTZ), hydroquinone (HQ), hydroquinone monomethyl ether (HQME), di-tert-butyl-para-cresol (BHT), para-phenylenediamine, TEMPO (2,2,6,6-tetramethyl-1-piperidinyloxy), di-tert-butylcatechol, or derivatives of TEMPO, such as OH-TEMPO, alone or as mixtures thereof in any proportions, at contents in the reaction medium which may be between 50 ppm and 5000 ppm, optionally in the presence of depleted air, but generally at contents of between 150 ppm and 1000 ppm. Polymerization inhibitors can be added at different locations, with the introduction of reagents or at the top of the distillation column.

To make the inhibitors more effective, it is appropriate to inject oxygen, air or so-called depleted air with 7% O2 at the bottom of the column. Preferably, the quantity of oxygen injected corresponds to a content of 0.2% to 0.5% relative to the quantity of organic vapor in the column.

The column can operate under vacuum to minimize thermal exposure of heat-sensitive compounds within the column. Advantageously, the topping column operates under a vacuum ranging from 1000 Pa to 30 000 Pa.

The bottom stream preferably feeds the column, making it possible to obtain the purified ester at the bottom of the column between theoretical trays 6 to 9.

The pure product distillation column comprises an equivalent of 2 and 15 theoretical trays, preferably 6 to 12 theoretical stages. The inserts used for the column may be valve trays or perforated weir trays, crossflow trays such as Dual Flow Trays, Ripple Trays, Turbo Grid Shells, or ordered packing, for instance structured packing such as Mellapack 250X from Sulzer.

The top stream of the column consists of high-purity butyl acrylate having as specifications an ester purity greater than 99.5%, a dibutyl ether content of less than 500 ppm, and finally a water content of less than 400 ppm.

The column operates with a reflux ratio (flow rate of condensed liquid returned to the column/flow rate of pure product) of between ⅛ to 1/1, preferably ¼. Like the topping column, this column is stabilized and air or depleted air (7% O2) is injected at the bottom of the column. The column can operate under vacuum to minimize thermal exposure of heat-sensitive compounds within the column. Advantageously, the pure product column operates under a vacuum ranging from 1000 pascal to 20 000 pascal.

Advantageously, the operating temperature is between 50° C. and 160° C.

The bottom stream is concentrated on an evaporator (not shown) with scraped film so as to recover and return to the rectification column feed the butyl acrylate which was at the bottom of this rectification column and to feed the cracker which is the subject of the invention with the bottom of the aforesaid. This residue is fed to a forced recirculation reactor comprising an external exchanger. The reaction temperature of the medium is between 220° and 300° C., preferably 230° C. to 280° C. The pressure in this reactor is maintained between 50 kPa and 300 kPa. The bottom product constitutes the final residue and is sent to the appropriate channel. The overhead product condensed at a temperature of 20° C. to 30° C. is sent to the inlet of the topping column. It is not necessary to inject air or depleted air into this reactor because the bottom product from the evaporator contains all the stabilizers used in the process.

The examples below illustrate the present invention without, however, limiting the scope thereof.

### EXPERIMENTAL SECTION

In the examples, the percentages are shown by weight, unless otherwise indicated, and the following abbreviations were used:
AA: acrylic acid
ABU: butyl acrylate
BuOH: butanol
BPB: butyl butoxypropionate
DBE: dibutyl ether

### Example 1 Catalytic Cracking Process According to Patent FR290172

A glass thermosiphon boiler with a useful capacity of 92 cm³ is used.

The boiler is fed continuously from an ABU heavy products vessel previously distilled by means of a diaphragm pump equipped with a back-pressure valve. The feed stream is sent to the boiler at room temperature and the pressure is maintained at atmospheric pressure. The feed flow rate is regulated by continuous measurement of the mass of initial mixture. The boiler is heated by means of 3 heating collars. A thermowell of 10 mm diameter measures the temperature in the reaction medium. The heating power is adjusted so as to have the desired temperature in the boiler. The vapors leaving the cracker are directed to a water-cooled condenser and the distillate is directed to an atmospheric pressure recovery tank.

For a composition comprising 70.5% BPB, 10.7% ABU catalyzed by 4.3% sulfuric acid, a residence time of 30 mins and a temperature of 171° C. in the cracker, the dibutyl ether content is 8% in the overhead product from the cracker. The reactor is clean.

### Example 2 Catalytic Cracking Process

A forced recirculation boiler with a volume of 40 1 is used, fed continuously by means of a diaphragm pump with ABU heavy products placed on a balance. The feed flow rate is measured by means of a mass flow meter placed on the feed line and also by the change in the mass indicated by the balance over time. The operation is carried out at atmospheric pressure. The temperature of the reaction medium and that at the inlet and outlet of the exchanger are measured continuously. The heat transfer fluid to bring the heat energies to the exchanger comes from an oil boiler. The heating power has been fixed so as to have 65% evaporation flow rate.

Under the following operating conditions: mass content of para-toluenesulfonic acid in the feed: 1.5%; atmospheric P; oil boiler T: 200° C., a residence time of 10 h expressed as the ratio of the reaction loop/feed flow rate, the distillate content is 65% and the latter comprises 1.4% dibutyl ether. In addition, the bottom product sample comprises solid particles.

### Example 3 Thermal Cracking Process According to the Invention

A forced recirculation boiler with a volume of 40 1 is used, fed continuously by means of a diaphragm pump with ABU heavy products placed on a balance. The feed flow rate is measured by means of a mass flow meter placed on the feed line and also by the change in the mass indicated by the balance over time. The operation is carried out at a pressure that is adjusted so as not to vaporize the butyl butoxypropionate. The temperature of the reaction medium and that at the inlet and outlet of the exchanger are measured continuously. The heat transfer fluid to bring the heat energies to the exchanger comes from an oil boiler. The heating power is fixed in order to keep the test temperature fixed.

The ABU heavy products were distilled beforehand under vacuum and contain about 2000 ppm of phenothiazine.

Table 2 below shows the results obtained:

TABLE 2

| Tests | 1 | 2 | 3 | 4 |
|---|---|---|---|---|
| T reactor (° C.) | 230 | 235 | 240 | 245 |
| Time (h) | 10 | 10 | 10 | 10 |
| Evaporation rate (%) | 41 | 43 | 47 | 50 |
| % DBE (ppm) | 2300 | 1356 | 1202 | 1050 |

Under these operating conditions, the dibutyl ether content is much lower than those stated in examples 1 and 2. The bottom product is clear.

1. A process for producing butyl acrylate by direct esterification of acrylic acid with butanol in the presence of sulfuric acid as catalyst, resulting in production of a crude reaction mixture containing butyl acrylate, residual acrylic acid and residual butanol, butyl hydrogen sulfate, traces of sulfuric acid and impurities resulting from side reactions, said process comprising steps of neutralizing and washing with water leading to the production of a reaction mixture free of acidic impurities, wherein said reaction mixture washed of acidic impurities is subjected at least to the following steps:
i) topping in a distillation column to obtain:
at the top, a stream composed essentially of unreacted reagents;
at the bottom, a stream comprising the desired ester and heavy by-products;
ii) subjecting the bottom stream from the topping column to a rectification column to separate:
at the top, purified butyl acrylate;
at the bottom, a stream containing heavy by-products, which is concentrated on a film evaporator or distilled in a tailing column in order to recycle the light compounds present to the rectification column feed, and to remove a final residue of heavy by-products;
iii) subjecting the bottom stream from the rectification column to a heat treatment carried out in the absence of catalyst in a cracker placed at the outlet of the evaporator, to separate:
at the top, a stream of upgradable products recycled to the topping column feed;
at the bottom, a residue sent to a treatment plant.

2. The process as claimed in claim 1, wherein said acidic impurities are sulfuric acid, butyl hydrogen sulfate, acrylic acid dimer and residual acrylic acid.

3. The process as claimed in claim 1, wherein the cracking is carried out at a temperature of 220° C. to 300° C.

4. The process as claimed in claim 1, wherein the cracking is carried out at a pressure of between 50,000 Pa to 300,000 Pa.

5. The process as claimed in claim 1, wherein the cracking is carried out in a continuous mode.

6. The process as claimed in claim 1, wherein the cracker is a tubular reactor, a jacketed stirred reactor or a reactor having an external heating loop with forced circulation.

7. The process as claimed in claim 1, wherein the butyl acrylate obtained has a purity of greater than 99.5%, a dibutyl ether content of less than 500 ppm, and a water content of less than 400 ppm.
